(19) United States
(12) Patent Application Publication
(10) Pub. No.: US 2021/0179585 A1
(43) Pub. Date: Jun. 17, 2021

(54) CRYSTAL FORM OF ABEMACICLIB MESYLATE, PREPARATION METHOD THEREFOR AND PHARMACEUTICAL COMPOSITION THEREOF

(71) Applicant: Hangzhou SoliPharma Co., Ltd., Hangzhou, Zhejiang (CN)

(72) Inventors: Xiaohong SHENG, Hangzhou, Zhejiang (CN); Xiaoxia SHENG, Hangzhou, Zhejiang (CN)

(21) Appl. No.: 17/048,384

(22) PCT Filed: Apr. 16, 2018
(86) PCT No.: PCT/CN2018/083152

§ 371 Date: Oct. 16, 2020

(51) Int. Cl. C07D 401/14

(52) U.S. Cl. CPC C07D 401/14; C07B 2200/13

(57) ABSTRACT

Disclosed is a crystal form of abemaciclib mesylate, which has one or more improved properties compared with the known abemaciclib mesylate. Also involved are a method for preparing the crystal form of abemaciclib mesylate, a pharmaceutical composition and the use of same in the preparation of a drug for treating cancer diseases such as colorectal cancer, breast cancer, lung cancer, prostate cancer, glioblastoma, mantle cell lymphoma, chronic granulocytic leukemia and acute granulocytic leukemia.

### FIELD OF THE INVENTION

The present invention relates to the technical field of crystallization in pharmaceutical chemistry. Specifically, the present invention relates to the novel crystalline forms of abemaciclib mesylate and the preparation methods, pharmaceutical compositions and uses thereof.

### BACKGROUND OF THE INVENTION

Abemaciclib mesylate is a special inhibitor of CDK4 and CDK6 and can be used to treat cancer diseases such as colorectal cancer, breast cancer, lung cancer, prostate cancer, glioblastoma, mantle cell lymphoma, chronic myelocytic leukemia, acute myelocytic leukemia, etc. The chemical name of abemaciclib mesylate is N-(5-((4-ethylpiperazin-1-yl)methyl)pyridin-2-yl)-5-fluoro-4-(4-fluoro-1-isopropyl-2-methyl-1H-benzo[d]imidazol-6-yl)pyrimidin-2-amine methanesulfonate, and its chemical structural formula is as follows:

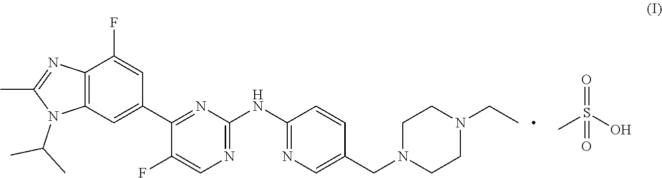

The patent CN102264725B disclosed the synthesis and preparation methods of abemaciclib mesylate; however, so far, there have been no reports of crystalline forms of abemaciclib mesylate. According to the preparation method of Embodiment 16 in CN102264725B, oily abemaciclib mesylate was obtained after removal of solvent under vacuum, and the abemaciclib mesylate solidified by grinding with methyl tert-butyl ether was amorphous. The appearance of oils and the necessity of grinding in order to solidify make it unlikely to industrialize the processing methods, and the obtained amorphous material has defects such as difficult to purify, poor chemical stability, high residue solvents and poor processability.

Therefore, it is still necessary to develop the crystalline forms of abemaciclib mesylate with improved properties to meet the strict requirements for active ingredients in pharmaceutical preparations.

### SUMMARY OF THE INVENTION

According to the defects in the prior art, the purpose of the present invention is to provide new crystalline forms of abemaciclib mesylate and their preparation methods, pharmaceutical compositions and uses thereof. The crystalline forms are stable crystalline solid with one or more improved properties, especially in the aspects of good crystallinity, good stability, good flowability, suitable processability, etc. In addition, the present invention relates to the preparation methods of the crystalline forms, and their pharmaceutical compositions and uses.

According to the purpose of the present invention, one aspect of the present invention is to provide the abemaciclib mesylate crystalline Form 1 (hereinafter referred to as “Form 1”).

In one embodiment of the present invention, measured using Cu-Kα radiation, the X-ray powder diffraction pattern of Form 1, expressed as 20 angles, has the following characteristic peaks: 4.0°±0.2°, 8.2°±0.2°, 23.6°±0.2° and 26.9°±0.2°.

In a preferred embodiment of the present invention, the X-ray powder diffraction pattern of Form 1, expressed as 20 angles, further has the following one or more characteristic peaks: 15.9°±0.2°, 16.6°±0.2°, 17.0°±0.2°, 18.5°±0.2° and 20.7°±0.2°.

In another preferred embodiment of the present invention, the X-ray powder diffraction pattern of Form 1, expressed as 20 angles, further has the following one or more characteristic peaks: 4.7°±0.2°, 9.5°±0.2°, 11.4°±0.2°, 12.7°±0.2°, 19.3°±0.2°, 22.5°±0.2° and 26.3°±0.2°.

In a further preferred embodiment of the present invention, the X-ray powder diffraction pattern of Form 1, expressed as 20 angles, further has the following one or more characteristic peaks: 10.5°±0.2°, 14.3°±0.2°, 15.3°±0.2°, 17.9°±0.2°, 18.3°±0.2°, 19.9°±0.2°, 20.2°±0.2°, 29.7°±0.2° and 30.5°±0.2°.

Non-restrictively, in one typical embodiment, the X-ray powder diffraction (XRPD) pattern of Form 1 is substantially as shown in FIG. 1.

Non-restrictively, the DSC thermogram of Form 1 is substantially as shown in FIG. 2.

Non-restrictively, the TGA thermogram of Form 1 is substantially as shown in FIG. 3.

Compared with the known amorphous abemaciclib mesylate, Form 1 of the present invention has the following beneficial properties:

1) Form 1 of the present invention is an anhydrous crystalline solid with better morphology.

2) Form 1 of the present invention kept its form unchanged for one month in a dry environment at room temperature and at 60° C. temperature, while weak crystalline state and discoloration appeared in the amorphous abemaciclib mesylate.

3) Form 1 of the present invention has a higher melting point.

The above advantageous properties of Form 1 show that, compared to the known amorphous abemaciclib mesylate, Form 1 of the present invention is a crystalline solid with better flowability and better processing characteristics (such as filtrating, drying, weighing, screening and so on during pharmaceutical manufacturing), which are beneficial in improving the homogeneity of formulations. In addition, Form 1 has good stability and higher melting point, which may better guarantee the quality, safety and stability of the active ingredient, ensure the quality, safety and stability of abemaciclib mesylate formulation containing Form 1 in its manufacturing and/or storage processes, avoid problems such as content uniformity issues of the active ingredients and increase in impurities, and also avoid special and expensive packaging.

According to the purpose of the present invention, the second aspect of the present invention is to provide the abemaciclib mesylate crystalline Form 2 (hereinafter referred to as “Form 2”).

In one embodiment of the present invention, measured using Cu-Kα radiation, the X-ray powder diffraction pattern of Form 2, expressed as 2θ angles, has the following characteristic peaks: 6.1°±0.2°, 12.0°±0.2°, 14.3°±0.2° and 21.6°±0.2°.

In a preferred embodiment of the present invention, the X-ray powder diffraction pattern of Form 2, expressed as 2θ angles, further has the following one or more characteristic peaks: 11.4°±0.2°, 12.7±0.2°, 13.0±0.2°, 14.7±0.2°, 15.6±0.2°, 17.5°±0.2°, 22.8°±0.2°, 23.4°±0.2°, 24.6°±0.2° and 26.9°±0.2°.

In a further preferred embodiment of the present invention, the X-ray powder diffraction pattern of Form 2, expressed as 2θ angles, further has the following one or more characteristic peaks: 11.2±0.2°, 15.1±0.2°, 18.0±0.2°, 20.0±0.2°, 23.1°±0.2°, 24.4°±0.2°, 25.7±0.2°, 27.5±0.2°, 28.6±0.2°, 29.6±0.2°, 30.2±0.2° and 32.6°±0.2°.

Non-restrictively, in one typical embodiment, the XRPD pattern of Form 2 is substantially as shown in FIG. 4.

Non-restrictively, the DSC thermogram of Form 2 is substantially as shown in FIG. 5.

Non-restrictively, the TGA thermogram of Form 2 is substantially as shown in FIG. 6.

Compared with the known amorphous abemaciclib mesylate, Form 2 of the present invention has the following beneficial properties:

1) Form 2 of the present invention is an anhydrous crystalline solid with better morphology.

2) Form 2 of the present invention kept its form unchanged for one month in a dry environment at room temperature and at 60° C., while weak crystalline state and discoloration appeared in the amorphous abemaciclib mesylate.

The above advantageous properties of Form 2 show that, compared to the known amorphous abemaciclib mesylate, Form 2 of the present invention is a crystalline solid with better flowability and better processing characteristics (such as filtrating, drying, weighing, screening and so on during pharmaceutical manufacture), which are beneficial in improving the homogeneity of pharmaceutical preparations. In addition, Form 2 has good stability which may better guarantee the quality, safety and stability of the active ingredient, ensure the quality, safety and stability of abemaciclib mesylate formulation containing Form 2 in its manufacturing and/or storage processes, avoid problems such as content uniformity issues of the active ingredients and increase in impurities, and also avoid special and expensive packaging.

According to the purpose of the present invention, the third aspect of the present invention is to provide abemaciclib mesylate crystalline Forms 3, 4, and 7 (hereinafter referred to as “Form 3”, “Form 4”, and “Form 7”).

Measured using Cu-Kα radiation, the X-ray powder diffraction pattern of Form 3, expressed as 2θ angles, has three or more of the following characteristic peaks: 8.1°±0.2°, 9.0°±0.2°, 11.7°±0.2°, 17.1°±0.2°, 17.9°±0.2°, 18.1°±0.2° and 21.7°±0.2°.

Measured using Cu-Kα radiation, the X-ray powder diffraction pattern of Form 4, expressed as 2θ angles, has three or more of the following characteristic peaks: 5.7°±0.2°, 6.4°±0.2°, 8.6±0.2°, 11.2°±0.2°, 14.3°±0.2°, 15.4°±0.2°, 16.7°±0.2°, 17.4±0.2°, 17.2±0.2°, 22.3±0.2°, 26.3°±0.2° and 27.1°±0.2°.

Measured using Cu-Kα radiation, the X-ray powder diffraction pattern of Form 7, expressed as 2θ angles, has three or more of the following characteristic peaks: 4.0°±0.2°, 14.5°±0.2°, 17.6°±0.2°, 18.0°±0.2°, 25.3°±0.2° and 27.7°±0.2°.

Compared with the known amorphous abemaciclib mesylate, Form 3, Form 4, and Form 7 of the present invention have one or more improved properties, such as higher crystallinity, better crystalline morphology, better thermal and storage stability, lower hygroscopicity, better flowability and more favorable processing and treatment properties.

According to the purpose of the present invention, the fourth aspect of the present invention is to provide the abemaciclib mesylate crystalline Form 5 (hereinafter referred to as “Form 5”).

In one embodiment of the present invention, measured using Cu-Kα radiation, the X-ray powder diffraction pattern of Form 5, expressed as 2θ angles, has the following characteristic peaks: 4.4°±0.2°, 9.0°±0.2°, 18.1±0.2° and 23.0°±0.2°.

In a preferred embodiment of the present invention, the X-ray powder diffraction pattern of Form 5, expressed as 2θ angles, further has the following one or more characteristic peaks: 11.8°±0.2°, 13.2±0.2°, 13.5°±0.2°, 16.3±0.2°, 20.6±0.2°, 21.7°±0.2°, 26.7°±0.2° and 30.7°±0.2°.

In a further preferred embodiment of the present invention, the X-ray powder diffraction pattern of Form 5, expressed as 2θ angles, further has the following one or more characteristic peaks: 14.2±0.2°, 14.5±0.2°, 15.4±0.2°, 17.1±0.2°, 18.2°±0.2°, 19.2°±0.2° and 22.1°±0.2°.

Non-restrictively, in one typical embodiment, the XRPD pattern of Form 5 is substantially as shown in FIG. 9.

Non-restrictively, the DSC thermogram of Form 5 is substantially as shown in FIG. 10.

Non-restrictively, the TGA thermogram of Form 5 is substantially as shown in FIG. 11.

Compared with the known amorphous abemaciclib mesylate, Form 5 of the present invention has the following beneficial properties:

1) Form 5 of the present invention is an anhydrous crystalline solid with better morphology.

2) Form 5 of the present invention kept its form unchanged for one month in a dry environment at 60° C. room temperature, while weak crystalline state and discoloration appeared in the amorphous abemaciclib mesylate.

The above advantageous properties of Form 5 show that, compared to the known amorphous abemaciclib mesylate, Form 5 of the present invention is a crystalline solid with better flowability and better processing characteristics (such as filtrating, drying, weighing, screening and so on during pharmaceutical manufacture), which are beneficial in improving the homogeneity of pharmaceutical preparations. In addition, Form 2 has good stability, which may better guarantee the quality, safety and stability of the active ingredient, ensure the quality, safety and stability of abemaciclib mesylate formulation containing Form 5 in its manufacturing and/or storage processes, avoid problems such as content uniformity issues of the active ingredients and increase in impurities, and also avoid special and expensive packaging.

According to the purpose of the present invention, the fifth aspect of the present invention is to provide the abemaciclib mesylate crystalline Form 6 (hereinafter referred to as “Form 6”).

In one embodiment of the present invention, measured using Cu-Kα radiation, the X-ray powder diffraction pattern of Form 6, expressed as 2θ angles, has the following characteristic peaks: 3.8°±0.2°, 7.5°±0.2°, 15.0°±0.2° and 18.8°±0.2°.

In a preferred embodiment of the present invention, the X-ray powder diffraction pattern of Form 6, expressed as 2θ angles, further has the following one or more characteristic peaks: 4.3°±0.2°, 8.6°±0.2°, 9.9±0.2°, 10.8±0.2°, 11.2±0.2°, 19.9±0.2°, 21.8°±0.2°, 22.6°±0.2°, 25.8°±0.2° and 28.8°±0.2°.

In a further preferred embodiment of the present invention, the X-ray powder diffraction pattern of Form 6, expressed as 2θ angles, further has the following one or more characteristic peaks: 12.9±0.2°, 16.4±0.2°, 17.2±0.2°, 18.3±0.2°, 19.2°±0.2°, 23.6°±0.2°, 24.5°±0.2°, 26.3°±0.2°, 27.2°±0.2°, 27.9°±0.2°, 34.2°±0.2° and 35.8°±0.2°.

Non-restrictively, in one typical embodiment, the XRPD pattern of Form 6 is subastantially as shown in FIG. 12.

Non-restrictively, the DSC thermogram of Form 6 is subastantially as shown in FIG. 13.

Non-restrictively, the TGA thermogram of Form 6 is subastantially as shown in FIG. 14.

Compared with the known amorphous abemaciclib mesylate, Form 6 of the present invention has the following beneficial properties:

1) Form 6 of the present invention is a semi-hydrate with better morphology.

2) Form 6 of the present invention kept its form unchanged for one month in a dry environment at room temperature and at 60° C., while weak crystalline state and discoloration appeared in amorphos abemaciclib mesylate.

The above advantageous properties of Form 6 show that, compared to the known amorphous abemaciclib mesylate, Form 6 of the present invention is a crystalline solid with better flowability and better subsequent processing characteristics (such as filtrating, drying, weighing, screening and so on during pharmaceutical manufacture), which are beneficial in improving the homogeneity of pharmaceutical formulations. In addition, Form 6 has good stability, which may better guarantee the quality, safety and stability of the active ingredient, ensure the quality, safety and stability of abemaciclib mesylate formulation containing Form 6 in its manufacturing and/or storage processes, avoid problems such as content uniformity issues of the active ingredients and increase in impurities, and also avoid special and expensive packaging.

According to the purpose of the present invention, the sixth aspect of the present invention is to provide the abemaciclib mesylate crystalline Form 8 (hereinafter referred to as “Form 8”).

In one embodiment of the present invention, measured using Cu-Kα radiation, the X-ray powder diffraction pattern of Form 8, expressed as 2θ angles, has the following characteristic peaks: 4.8°±0.2°, 9.5°±0.2°, 14.2°±0.2° and 22.5±0.2°.

In a preferred embodiment of the present invention, the X-ray powder diffraction pattern of Form 8, expressed as 2θ angles, further has the following one or more characteristic peaks: 11.9°±0.2°, 16.8±0.2°, 18.6±0.2°, 23.7±0.2°, 24.8°±0.2° and 26.2°±0.2°.

In a further preferred embodiment of the present invention, the X-ray powder diffraction pattern of Form 8, expressed as 2θ angles, further has the following one or more characteristic peaks: 10.1±0.2°, 17.1±0.2°, 18.9±0.2°, 19.8±0.2°, 20.3°±0.2°, 20.8°±0.2°, 24.3°±0.2°, 28.2°±0.2°, 29.7°±0.2° and 30.3°±0.2°.

Non-restrictively, in one typical embodiment, the XRPD pattern of Form 8 is subastantially as shown in FIG. 16.

Non-restrictively, the DSC thermogram of Form 8 is subastantially as shown in FIG. 17.

Non-restrictively, the TGA thermogram of Form 8 is subastantially as shown in FIG. 18.

Compared with the known amorphous abemaciclib mesylate, Form 8 of the present invention has the following beneficial properties:

1) Form 8 of the present invention is an anhydrous crystalline solid with better morphology.

2) Form 8 of the present invention kept its form unchanged for one month in a dry environment at room temperature and at 60° C., while weak crystalline state and discoloration appeared in the amorphous abemaciclib mesylate.

The above advantageous properties of Form 8 show that, compared to the known amorphous forms, Form 8 of the present invention is a crystalline solid with better flowability and better subsequent processing characteristics (such as filtrating, drying, weighing, screening and so on during pharmaceutical manufacture), which are beneficial in improving the homogeneity of pharmaceutical preparations. In addition, the Form 8 has good stability, which may better guarantee the quality, safety and stability of the active ingredient, ensure the quality, safety and stability of abemaciclib mesylate formulation containing the Form 8 in its manufacturing and/or storage processes, avoid problems such as content uniformity issues of the active ingredients and increase in impurities, and also avoid special and expensive packaging.

According to the purpose of the present invention, the seventh aspect of the present invention is to provide preparation methods of the abemaciclib mesylate crystalline Form 1, Form 2, Form 5, Form 6, and Form 8.

The preparation methods of Form 1 comprise any of the following preparation methods:

1) dissolving abemaciclib in a haloalkane solvent to form a clear solution, stirring and dripping in methanesulfonic acid, adding an anti-solvent while stirring for crystallization, filtrating and drying the solid to obtain the abemaciclib mesylate crystalline Form 1:

preferably, the haloalkane solvent is dichloromethane;

preferably, the molar ratio of abemaciclib and methanesulfonic acid is 1:1 to 1:1.5;

preferably, the anti-solvent is isopropyl ether, in an amount 2 to 4 times that of dichloromethane;

preferably, the mass-to-volume ratio of abemaciclib to the solvent is 20 to 170 mg:1 mL, more preferably, 40 to 170 mg:1 mL;

preferably, the reaction is carried out at room temperature;

preferably, the stirring crystallization time is 3 to 5 days;

preferably, the drying temperature is 25 to 60° C.;

preferably, the drying time is 16 to 48 hours; or

2) dissolving abemaciclib in a haloalkane solvent to form a clear solution, stirring and then dripping in methanesulfonic acid, stirring for crystallization, filtrating and drying the solid to obtain the abemaciclib mesylate crystalline Form 1:

preferably, the haloalkane solvent is dichloromethane;

preferably, the molar ratio of abemaciclib and methanesulfonic acid is 1:1 to 1:1.5;

preferably, the mass-to-volume ratio of abemaciclib to the solvent is 50 to 250 mg:1 mL, more preferably, 60 to 250 mg:1 mL;

preferably, the reaction is carried out at room temperature;

preferably, the stirring crystallization time is 3 to 7 days;

preferably, the drying temperature is 25 to 60° C.;

preferably, the drying time is 16 to 48 hours.

The preparation method of Form 2 is as follows:

dissolving abemaciclib in a ketone solvent to form a suspension, stirring and then dripping in methanesulfonic acid, stirring for crystallization, filtrating and drying the solid to obtain the abemaciclib mesylate crystalline Form 2:

preferably, the ketone solvent is acetone;

preferably, the molar ratio of abemaciclib and methanesulfonic acid is 1:1 to 1:1.5;

preferably, the mass-to-volume ratio of abemaciclib to the solvent is 5 to 25 mg:1 mL, more preferably, 10 to 25 mg:1 mL;

preferably, the reaction is carried out at room temperature;

preferably, the stirring crystallization time is 3 to 7 days;

preferably, the drying temperature is 25 to 60° C.;

preferably, the drying time is 16 to 48 hours.

The preparation methods of Form 5 comprise any of the following preparation methods:

1) dissolving abemaciclib mesylate in a C₁ to C₄ alcohol, a C₃ to C₆ ether, a C₄ to C₅ ester, a C₃ to C₄ ketone, a cycloether, nitrile, water, an alkane, nitromethane, or any mixed solvent thereof to form a suspension, stirring for crystallization, separating and drying the solid to obtain the abemaciclib mesylate crystalline Form 5:

preferably, the mass-to-volume ratio of abemaciclib mesylate to the solvent is 15 to 100 mg:1 mL, more preferably, 20 to 50 mg:1 mL;

preferably, the reaction is carried out at 4 to 40° C.;

preferably, the stirring crystallization time is 3 to 7 days;

preferably, the drying temperature is 25 to 60° C.;

preferably, the drying time is 16 to 48 hours; or

2) dissolving abemaciclib mesylate in a C₁ to C₄ alcohol to form a clear solution, adding an anti-solvent while stirring for crystallization, filtrating and drying the solid to obtain abemaciclib mesylate Form 5:

preferably, the mass-to-volume ratio of the solid abemaciclib mesylate to the C₁ to C₄ alcohol is 10 to 35 mg:1 mL, more preferably, 10 to 20 mg:1 mL;

preferably, the anti-solvent is a C₃ to C₆ ether or a C₆ to C₇ alkane, in an amount 2 to 4 times of the C₁ to C₄ alcohol;

preferably, the reaction is carried out at room temperature;

preferably, the drying temperature is 25 to 60° C.;

preferably, the drying time is 16 to 48 hours.

The preparation methods of Form 6 comprise any of the following preparation methods:

1) placing a solid abemaciclib mesylate at a 58% to 85% humidity condition to obtain the abemaciclib mesylate crystalline Form 6;

preferably, the placing environment temperature is at 20 to 40° C.

2) dissolving a solid abemaciclib mesylate in ethanol, heating the solution to form a clear solution, stirring the solution at a low temperature for crystallization, filtrating and drying the solid to obtain the abemaciclib mesylate crystalline Form 6;

preferably, the mass-to-volume ratio of solid abemaciclib mesylate to ethanol is 20 to 25 mg:1 mL;

preferably, the low temperature is −10 to 5° C.;

preferably, the drying temperature is 25 to 60° C.;

preferably, the drying time is 16 to 48 hours.

The preparation method of Form 8 is as follows:

keeping the abemaciclib mesylate crystalline Form 1, Form 2, Form 5, or Form 6 at 200 to 210° C. for a certain period of time to obtain the abemaciclib mesylate crystalline Form 8.

In the above preparation methods of crystalline forms of abemaciclib mesylate of the present invention: the starting material “abemaciclib” may be obtained by referring to the method described in Embodiment 16 of the patent CN102264725(B) or commercially available. This patent document is incorporated into this application by reference in its entirety.

In the terms used in the present invention are as follows:

The term “room temperature” refers to a temperature between 10° C. and 30° C.

The “stirring” may be carried out by a conventional stirring method in the art, such as magnetic stirring, mechanical stirring and the stirring speed is 50 to 1,800 rpm, preferably 300 to 900 rpm.

The “separation” may be performed using conventional methods in the field, such as centrifugation or filtration. The preferred method is vacuum filtration, generally at a pressure less than atmosphere pressure at room temperature, preferably less than 0.09 Mpa.

The “drying” may be performed by routine methods in the field, such as room temperature drying, forced air drying or vacuum drying. Drying instruments and methods are unrestricted, and may be fume hood, blast oven, spray drying, fluidized bed drying or vacuum oven. Drying is performed under a reduced pressure or at atmospheric pressure, preferably less than 0.09 Mpa. Drying temperature is 10 to 40° C., the drying time is 10 to 72 hours, preferably 2 to 24 hours and more preferably 2 to 8 hours.

In the present invention, “crystal” or “crystalline form” refers to that characterized by X-ray powder diffraction pattern, having an unique ordered molecular arrangement or conformation within the crystalline lattice. It is known to those skilled in the field that the experimental error depends on instrumental conditions, sample preparation and sample purity. The 20 angles of the peaks in the XRPD pattern may change with the change of instruments and samples. The difference of peak positions may vary by 1°, 0.8°, 0.5°, 0.3°, 0.1°, etc. depending on the instruments and samples, and ±0.2° is usually allowed. Therefore, the difference in peak angles should not be regarded as the only factor. The relative intensity of peaks may change with the change of sample, sample preparation, and other experimental conditions. Therefore, the order of peak intensities should not be regarded as the only or the determining factor. Due to effect of experimental factors including sample height, the angles of the peaks may shift overall. Hence, it is easily understood for those skilled in the field that any crystalline form having the same or similar X-ray powder diffraction pattern as that of the crystalline form in the present invention should be within the scope of the present invention. “Single crystalline form” refers to a crystalline form confirmed by X-ray powder diffraction as a single form.

The abemaciclib mesylate crystalline forms of the present invention are substantially pure, single, and substantially free of any other crystalline or amorphous form. As used herein, “substantially pure” when used in reference to a new crystalline form means that the new crystalline form comprises at least 80% (by weight) of the present compound, more preferably at least 95% (by weight), especially at least 95% (by weight), especially at least 99% (by weight).

According to the purpose of the present invention, the eighth aspect of the present invention is to provide a pharmaceutical composition, which comprises a therapeutic effective amount of active pharmaceutical ingredient selected from the group consisting of crystalline forms of abemaciclib mesylate of the present invention or from the crystalline forms of abemaciclib mesylate prepared with the preparation methods of the present invention, and at least one pharmaceutically acceptable carrier or excipient. The crystalline forms of abemaciclib mesylate of the present invention include Form 1, Form 2, Form 3, Form 4, Form 5, Form 6, Form 7 and Form 8. In addition, the pharmaceutical composition may also comprise other pharmaceutical crystalline forms and amorphous forms. The dosage form of the compound used in the method of the present invention can be determined by selected specific solid state of the compound, the administration route and patient status. Using generally accepted methods in the field, the crystalline forms of the present invention can be prepared to a dosage form suitable for one or more of the following administration routes, which include oral administration, sublingual administration, parenteral injection (including subcutaneous injection, intravenous injection, intramuscular injection, sternum injection or infusion technology), inhalation administration, nasal administration, or rectum administration, and the pharmaceutical formulation contains at least one active ingredient. Preferably, the pharmaceutical composition of the present invention may be provided in individual dose unit, and each dosage may contain abemaciclib mesylate of the present invention and its crystalline forms from 5 mg to 1,000 mg approximately, more often, from 100 mg to 500 mg approximately.

The term of “patient” used herein refers to an animal that is the target of treatment, observation or experiments, preferably a mammal, more preferably a human.

The term “treatment” used herein refers to one or more of the following circumstances: 1) prevention of disease and its symptoms; for example, doing so in the patients who may be inclined to develop a disease or symptoms but have not suffered from or shown a lesion or symptoms of the disease; 2) inhibition of the disease and symptoms; for example, doing so in the patients who are suffering from or showing a disease or its symptoms; and 3) improvement of the disease; for example, doing so in the patients who suffer from or show a disease or its symptoms (i.e. reversing the disease and/or its symptoms), for example, reducing the severity of the disease.

The term “or” used herein refers to standby options, which can be combined if appropriate. Therefore, the term “or” contains the options listed separately and their combinations, as long as the combinations are not mutually exclusive.

The term “effective dose” refers to a dose sufficient to perform a therapeutic or preventive function. “Pharmacologically acceptable” means that the ingredients of the pharmaceutical composition are compatible with each other and are suitable for the recipient.

The “pharmaceutical composition” or “composition” is intended to include a bulk composition and/or individual dose unit consisting of one or more active pharmaceutical ingredients of the abemaciclib mesylate crystalline forms of the present invention and any non-pharmaceutically active excipients. Bulk compositions and individual dose units may contain a fixed amount of one or more of the above active pharmaceutical ingredients. Bulk compositions are substances that have not yet formed individual units of dose. Examples of nonrestrictive dose units are oral dose units for tablets, pills, and their analogues. Similarly, the pharmaceutical composition of the present invention given to the individual in need is intended to include both bulk composition and individual dose unit mentioned above.

Generally, the composition of the present invention comprises an active ingredient, which is usually mixed with an excipient, diluted by an excipient, or enclosed within such a carrier which can be in a form of a capsule, a sachet or other container. When an excipient is used as a diluent, it may be a solid, semi-solid, or liquid material, which functions as an excipient, carrier or medium of the active ingredient. Thus, the pharmaceutical composition may be in the form of tablets, pills, lozenges, powders, sachets, cachets, elixirs, suspensions, emulsions, solutions, syrups, aerosols (either in solid or in liquid medium), sprays, ointments, soft or hard capsules, gels, suppositories, sterile injectable solutions and sterile package powders.

Examples of excipients for solid dosage form suitable for oral administration include sugars, such as glucose, sucrose, lactose, sorbitol, mannose, starch, gum Arabic, calcium phosphate, alginate, gelatin, calcium silicate, microcrystalline cellulose, polyvinylpyrrolidone, kaolin, cellulose, methyle cellulose, adhesives and disintegrating agents. Some liquid formulations suitable oral administration (such as suspending agents, syrups, elixirs, etc.) can use media such as water, glycol, oil or alcohol. Parenteral composition usually uses sterile water as a carrier and optionally other ingredients such as solubilizers. Injectable solution may be prepared, for example, using a carrier containing a saline solution, a glucose solution or a mixture of saline and glucose. Pharmaceutical formulation may also include lubricants such as talc powder, magnesium stearate and mineral oils, wetting agents, emulsifiers and suspensions, preservatives such as propyl hydroxybenzoate, sweeteners and flavoring agents. The compound of the present invention can be prepared by the methods known in this field, so that the active ingredient can be released rapidly, continuously or delayed after drug administration.

According to the purpose of the present invention, the ninth aspect of the present invention is to provide the uses of the crystalline forms of the present invention or crystalline forms obtained using the preparation methods of the present invention in the preparation of the drugs treating cancer diseases such as colorectal cancer, breast cancer, lung cancer, prostate cancer, glioblastoma, mantle cell lymphoma, chronic myelocytic leukemia, acute myelocytic leukemia, etc.

According to the purpose of the present invention, the invention provides methods for treating cancer diseases such as colorectal cancer, breast cancer, lung cancer, prostate cancer, glioblastoma, mantle cell lymphoma, chronic myelocytic leukemia, acute myelocytic leukemia, etc. and their symptoms, comprising administering to a patient in need a pharmaceutical composition containing an effective amount abemaciclib mesylate selected from Form 1, Form 2, Form 3, Form 4, Form 5, Form 6, Form 7 and Form 8 of the present invention. The diseases are the same as those described above.

The active compound is usually effective in a large dose range. For example, the daily dose of the active ingredient (either single dose or factional dose) is generally about 1 μg to 1 g per kg body weight per day. For the treatment of adult, the preferred dose (single dose or factional dose) is approximately 0.01 mg to 100 mg per kg body weight per day. However, it should be understood that the actual amount and frequency of drug administration for any given patient vary and depend on a variety of factors, including the potency of the compound used, metabolism and duration of drug action, one or more compounds actually to be taken, the disease to be treated and its severity, administration route, age, weight, excretion rate and overall response of the specific patient, thus the above dose range shall not limit the scope of the invention in any way. In some cases, a dose level below the above said range may be more appropriate, while in others a larger dose without any side effects may be used, provided that the larger dose is first divided into smaller doses for the full day to use.

### DESCRIPTION OF DRAWINGS

FIG. 1 is the XRPD pattern of abemaciclib mesylate Form 1 of the present invention.

FIG. 2 is the DSC thermogram of abemaciclib mesylate Form 1 of the present invention.

FIG. 3 is the TGA thermogram of abemaciclib mesylate Form 1 of the present invention.

FIG. 4 is the XRPD pattern of abemaciclib mesylate Form 2 of the present invention.

FIG. 5 is the DSC thermogram of abemaciclib mesylate Form 2 of the present invention.

FIG. 6 is the TGA thermogram of abemaciclib mesylate Form 2 of the present invention.

FIG. 7 is the XRPD pattern of abemaciclib mesylate Form 3 of the present invention.

FIG. 8 is the XRPD pattern of abemaciclib mesylate Form 4 of the present invention.

FIG. 9 is the XRPD pattern of abemaciclib mesylate Form 5 of the present invention.

FIG. 10 is the DSC thermogram of abemaciclib mesylate Form 5 of the present invention.

FIG. 11 is the TGA thermogram of abemaciclib mesylate Form 5 of the present invention.

FIG. 12 is the XRPD pattern of abemaciclib mesylate Form 6 of the present invention.

FIG. 13 is the DSC thermogram of abemaciclib mesylate Form 6 of the present invention.

FIG. 14 is the TGA thermogram of abemaciclib mesylate Form 6 of the present invention.

FIG. 15 is the XRPD pattern of abemaciclib mesylate Form 7 of the present invention.

FIG. 16 is the XRPD pattern of abemaciclib mesylate Form 8 of the present invention.

FIG. 17 is the DSC thermogram of abemaciclib mesylate Form 8 of the present invention.

FIG. 18 is the TGA thermogram of abemaciclib mesylate Form 8 of the present invention.

### SPECIFIC IMPLEMENTATIONS

The following examples help to further understand the present invention, but are not intended to limit the contents of the present invention.

### Testing Instruments and Methods:

X-ray powder diffraction (XRPD): performed on a Bruker D8 Advance diffractometer. Samples are tested at room temperature. Testing conditions: 2θ scan range 3 to 40°, step size 0.02° and speed 0.2 s/step.

Differential scanning calorimetry (DSC) analysis data were collected on a TA Instruments Q200 DSC. Method: A sample of 1 to 10 mg was placed in a sealed aluminum pan, and the sample was heated from room temperature to 250° C. at a heating rate of 10° C./min under the protection of dry nitrogen purge at 40 mL/min.

Thermogravimetric (TGA) analysis data were collected on a TA Instruments Q500 TGA. Method: A sample of 5 to 15 mg was placed in a platinum pan, using High Resolution™, the sample was heated from room temperature to 350° C. at a heating rate of 10° C./min under the protection of dry nitrogen purge at 40 mL/min.

¹H nuclear magnetic resonance (1H-NMR) data were collected on a Bruker Ascend Tm 500 nuclear magnetic resonance spectrometer using full frequency excitation with spectrum width 30 ppm, single pulse, 30° angle excitation, scanning 16 times, digital orthogonal testing and temperature control 298K.

Unless particularly specified, all reagents used in the embodiments were commercially available.

Unless particularly specified, all embodiments were operated at room temperature.

### Preparation Example 1

Abemaciclib was prepared by referring the method in Example 1 in the patent CN102264725(B).

### Preparation Example 2

Abemaciclib mesylate was prepared by referring the method in Example 16 in the patent CN102264725(B). The specific operation was as follows: Removing solvent under vacuum to obtain an oily sample, adding methyl tert-butyl ether (MTBE) and grinding to obtain amorphous abemaciclib mesylate.

### Example 1

500 mg abemaciclib was dissolved in 2 mL dichloromethane to form a clear solution, the solution was stirred at room temperature and 1 mL dichloromethane solution containing 96.2 mg methanesulfonic acid was dripped in, then 5 mL isopropyl ether was added, white solid appeared; 3 days after stirring, the obtained solid was filtrated and vacuum dried at 40° C., and abemaciclib mesylate Form 1 was obtained.

Its XRPD pattern is shown in FIG. 1.

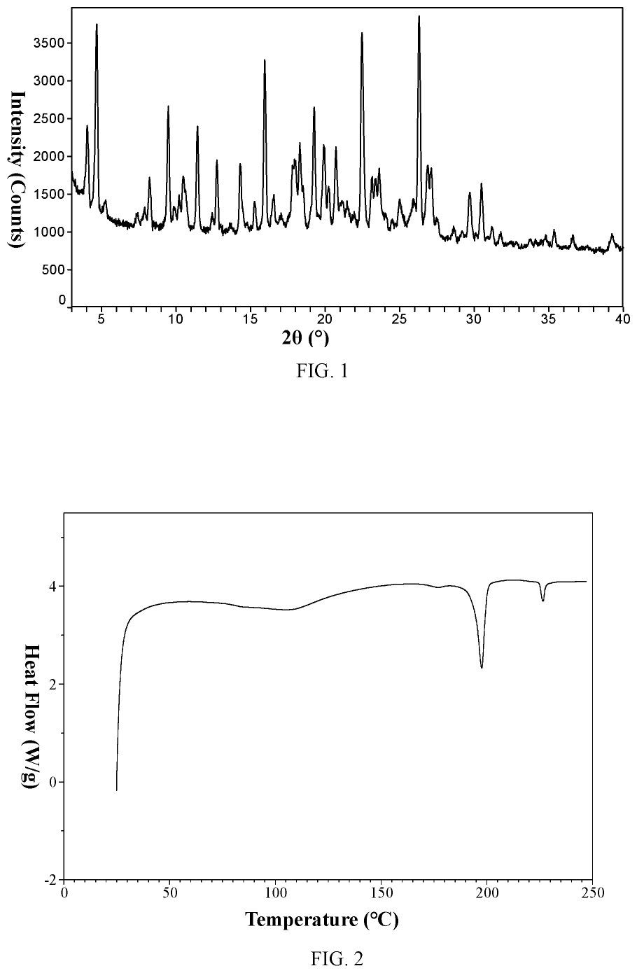

Its DSC thermogram is shown in FIG. 2.

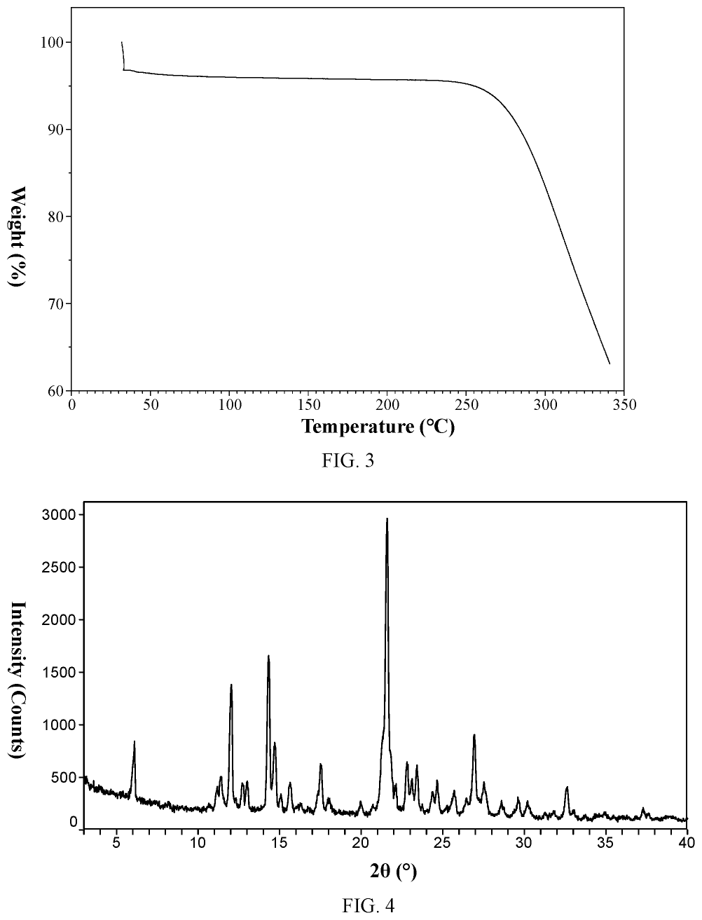

Its TGA thermogram is shown in FIG. 3.

The ¹H-NMR (DMSO) data of the product are as follows:

¹H-NMR (500 MHz, DMSO-d6): δ10.18 (s, 1H), 8.71 (d, J=3.7 Hz, 1H), 8.28 (s, 1H), 8.27-8.20 (m, 2H), 7.71 (t, J=10.3 Hz, 2H), 4.85 (hept, J=6.9 Hz, 1H), 3.59 (s, 2H), 3.13 (s, 2H), 3.04-2.89 (m, 4H), 2.65 (s, 3H), 2.33 (s, 3H), 1.63 (d, J=6.9 Hz, 6H), 1.21 (t, J=7.3 Hz, 3H). It indicates that the compound is abemaciclib mesylate.

### Example 2

500 mg abemaciclib was dissolved in 2 mL dichloromethane to form a clear solution, the solution was stirred at room temperature and 1 mL dichloromethane solution containing 96.2 mg methanesulfonic acid was dripped in, 5 min after stirring, white solid appeared; 7 days after continuous stirring, the obtained solid was filtrated and vacuum dried at 25° C. for 48 hours, and abemaciclib mesylate Form 1 was obtained.

### Example 3

250 mg abemaciclib was dissolved in 6.25 mL dichloromethane to form a clear solution, the solution was stirred at room temperature and 1 mL dichloromethane solution containing 71 mg methanesulfonic acid was dripped in, then 12.5 mL isopropyl ether was added, white solid appeared; 3 days after stirring, the solid was filtrated and vacuum dried at 60° C. for 16 hours, and abemaciclib mesylate Form 1 was obtained.

### Example 4

250 mg abemaciclib was dissolved in 12.5 mL dichloromethane to form a clear solution, the solution was stirred at room temperature and 1 mL dichloromethane solution containing 47 mg methanesulfonic acid was dripped in, then 30 mL isopropyl ether was added, white solid appeared; 5 days after stirring, the solid was filtrated and vacuum dried at 25° C. for 48 hours, and abemaciclib mesylate Form 1 was obtained.

### Example 5

250 mg abemaciclib was dissolved in 5 mL dichloromethane to form a clear solution, the solution was stirred at room temperature and 1 mL dichloromethane solution containing 47.4 mg methanesulfonic acid was dripped in, 5 minutes after stirring, white solid appeared; 3 days after stirring, the solid was filtrated and vacuum dried at 60° C. for 16 hours, and abemaciclib mesylate Form 1 was obtained.

### Example 6

250 mg abemaciclib was dissolved in 4.2 mL dichloromethane to form a clear solution, the solution was stirred at room temperature and 1 mL dichloromethane solution containing 96.2 mg methanesulfonic acid was dripped in, 5 minutes after stirring, white solids precipated; 5 days after stirring, the solid was filtrated and vacuum dried at 40° C. for 24 hours, and abemaciclib mesylate Form 1 was obtained.

The samples prepared in Examples 2 to 6 have essentially the same or similar XRPD patterns (not shown), DSC thermograms (not shown), and TGA thermograms (not shown) as those of the sample of Example 1, indicating that the samples of Examples 2 to 6 and Example 1 have the same crystalline form.

### Example 7

250 mg abemaciclib was dissolved in 10 mL acetone, the solution was stirred at room temperature and 3 mL acetone solution containing 47.4 mg methanesulfonic acid was dripped in; 7 days after stirring, the solid was filtrated and vacuum dried at 60° C. for 48 hours, and abemaciclib mesylate Form 2 was obtained.

Its XRPD pattern is shown in FIG. 4.

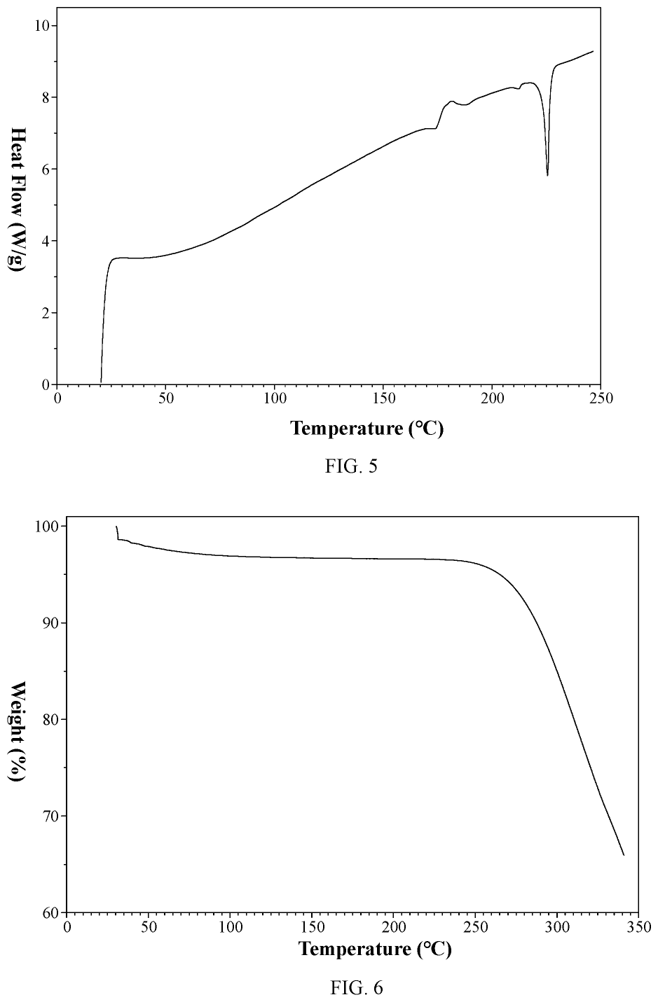

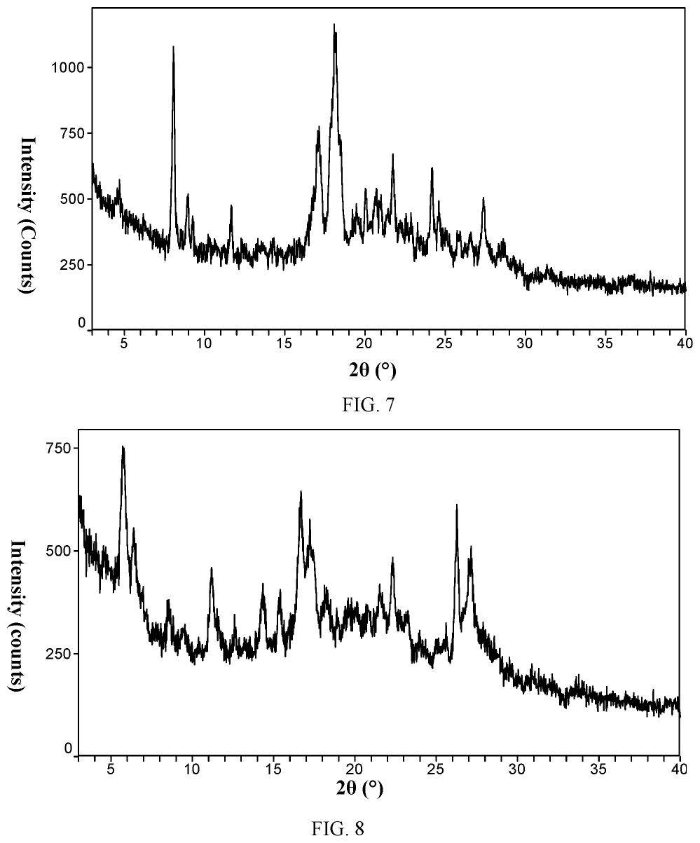

Its DSC thermogram is shown in FIG. 5.

Its TGA thermogram is shown in FIG. 6.

The 1H-NMR (DMSO) data of the product are as follows:

¹H-NMR (500 MHz, DMSO-d6): δ9.17 (s, 1H), 8.71 (d, J=3.7 Hz, 1H), 8.37-8.11 (m, 3H), 7.71 (t, J=11.4 Hz, 2H), 4.84 (h, J=7.0 Hz, 1H), 3.59 (s, 2H), 3.12 (d, J=8.4 Hz, 2H), 2.97 (s, 4H), 2.65 (s, 3H), 2.33 (s, 4H), 1.63 (d, J=6.9 Hz, 6H), 1.21 (t, J=7.3 Hz, 3H). It indicates that the compound is abemaciclib mesylate.

### Example 8

50 mg abemaciclib was dissolved in 10 mL acetone, the solution was stirred at room temperature and 2 mL acetone solution containing 10 mg methanesulfonic acid was dripped in; 3 days after stirring, the solid was filtrated and vacuum dried at 25° C. for 16 hours, and abemaciclib mesylate Form 2 was obtained.

### Example 9

50 mg abemaciclib was dissolved in 5 mL acetone, the solution was stirred at room temperature and 2 mL acetone solution containing 14.2 mg methanesulfonic acid was dripped in; 5 days after stirring, the solid was filtrated and vacuum dried at 40° C. for 24 hours, and abemaciclib mesylate Form 2 was obtained.

The samples prepared in Examples 8 to 9 have essentially the same or similar XRPD patterns (not shown), TGA thermograms (not shown) as those of the sample of Example 7, indicating that the samples of Examples 8 to 9 and Example 7 have the same crystalline form.

### Example 10

100 mg abemaciclib mesylate in Preparation Example 2 was dissolved in 10 mL ethanol to form a suspension, and it was stirred at 4° C. for 7 days, the solid was filtrated and vacuum dried at 60° C. for 48 hours, and abemaciclib mesylate Form 5 was obtained.

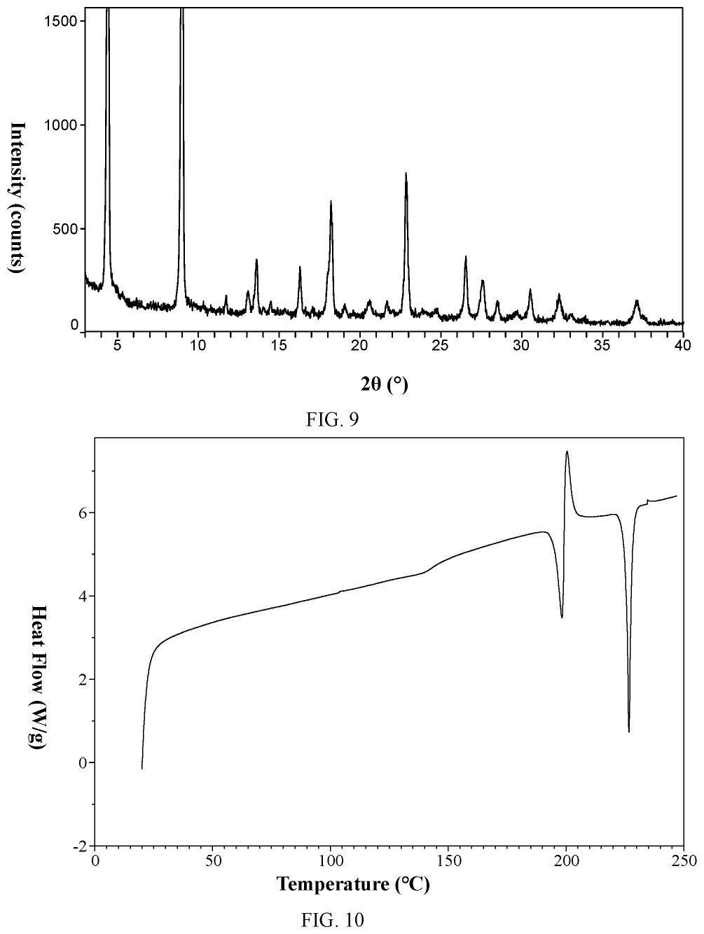

Its XRPD pattern is shown in FIG. 9.

Its DSC thermogram is shown in FIG. 10.

Its TGA thermogram is shown in FIG. 11.

The 1H-NMR (DMSO)data of the product are as follows:

1HNMR (500 MHz, DMSO-d6): δ10.12 (s, 1H), 8.68 (d, J=3.6 Hz, 1H), 8.46-8.00 (m, 3H), 7.68 (d, J=12.5 Hz, 2H), 4.83 (p, J=7.0 Hz, 1H), 3.56 (s, 2H), 3.11 (d, J=8.2 Hz, 2H), 2.94 (d, J=13.2 Hz, 4H), 2.63 (s, 3H), 2.31 (d, J=2.7 Hz, 5H), 1.61 (d, J=6.8 Hz, 6H), 1.19 (t, J=7.3 Hz, 3H). It indicates that the compound is abemaciclib mesylate.

### Example 11

20 mg abemaciclib mesylate in Preparation Example 2 was dissolved in 0.2 mL methanol and 0.8 mL isopropyl acetate to form a suspension, and it was stirred at 25° C. for 5 days, the solid was then filtrated and vacuum dried at 40° C. for 16 hours, and abemaciclib mesylate Form 5 was obtained.

### Example 12

15 mg abemaciclib mesylate in Preparation Example 2 was dissolved in 1 mL methyl tert-butyl ether to form a suspension, and it was stirred at 25° C. for 7 days, the solid was filtrated and vacuum dried at 60° C. for 24 hours, and abemaciclib mesylate Form 5 was obtained.

### Example 13

20 mg abemaciclib mesylate in Preparation Example 2 was dissolved in 0.4 mL acetone to form a suspension, and it was stirred at 4° C. for 3 days, the solid was filtrated and vacuum dried at 60° C. for 16 hours, and abemaciclib mesylate Form 5 was obtained.

### Example 14

20 mg abemaciclib mesylate in Preparation Example 2 was dissolved in 0.6 mL tetramethylene oxide to form a suspension, and it was stirred at 25° C. for 7 days, the solid was filtrated and vacuum dried at 40° C. for 24 hours, and abemaciclib mesylate Form 5 was obtained.

### Example 15

20 mg abemaciclib mesylate in Preparation Example 2 was dissolved in 0.8 mL 1,4-dioxane to form a suspension, and it was stirred at 25° C. for 7 days, the solid was filtrated and vacuum dried at 40° C. for 24 hours, and abemaciclib mesylate Form 5 was obtained.

### Example 16

20 mg abemaciclib mesylate in Preparation Example 2 was dissolved in 0.4 mL acetonitrile and 0.1 mL water to form a suspension, and it was stirred at 4° C. for 7 days, the solid was filtrated and vacuum dried at 40° C. for 24 hours, and abemaciclib mesylate Form 5 was obtained.

### Example 17

20 mg abemaciclib mesylate in Preparation Example 2 was dissolved in 0.2 mL nitromethane, 0.2 mL butanone and 0.2 mL 1,4-dioxane to form a suspension, and it was stirred at 25° C. for 7 days, the solid was filtrated and vacuum dried at 40° C. for 24 hours, and abemaciclib mesylate Form 5 was obtained.

### Example 18

20 mg abemaciclib mesylate in Preparation Example 2 was dissolved in 0.2 mL 2-butanol, 0.2 mL ethyl acetate and 0.2 mL chloroform to form a suspension, and it was stirred at 25° C. for 7 days, the solid was filtrated and vacuum dried at 40° C. for 24 hours, and abemaciclib mesylate Form 5 was obtained.

### Example 19

20 mg abemaciclib mesylate in Preparation Example 2 was dissolved in 0.6 mL butanone and 0.6 mL n-heptane to form a suspension, then it was stirred at 40° C. for 3 days, the solid was filtrated and vacuum dried at 40° C. for 24 hours, and abemaciclib mesylate Form 5 was obtained.

### Example 20

20 mg abemaciclib mesylate in Preparation Example 2 was dissolved in 0.2 mL n-butanol, 0.6 mL isopropyl ether and 0.2 mL ethyl acetate to form a suspension, then it was stirred at 25° C. for 7 days, the solid was filtrated and vacuum dried at 40° C. for 24 hours, and abemaciclib mesylate Form 5 was obtained.

### Example 21

20 mg abemaciclib mesylate in Preparation Example 2 was dissolved in 0.6 mL methanol to form a clear solution, under stirring added with 2.4 mL isopropyl ether, white turbidity appeared; the solid was filtrated and vacuum dried at 25° C. for 48 hours, and abemaciclib mesylate Form 5 was obtained.

### Example 22

20 mg abemaciclib mesylate in Preparation Example 2 was dissolved in 10 mL ethanol to form a clear solution, under stirring added with 3 mL n-heptane, white turbidity appeared; the solid was filtrated and vacuum dried at 60° C. for 16 hours, and abemaciclib mesylate Form 5 was obtained.

### Example 23

20 mg abemaciclib mesylate in Preparation Example 2 was dissolved in 2 mL 2-butanol to form a clear solution, under stirring added with 4 mL methyl tert-butyl ether, white turbidity appeared; the solid was filtrated and vacuum dried at 40° C. for 24 hours, and abemaciclib mesylate Form 5 was obtained.

The samples prepared in Examples 11 to 23 essentially have the same or similar XRPD patterns (not shown), TGA thermograms (not shown) as those of the sample of Example 10, indicating that the samples of Examples 11 to 23 and Example 10 have the same crystalline form.

### Example 24

100 mg abemaciclib mesylate in Preparation Example 2 was dissolved in 4 mL ethanol, and it was heated at 60° C. to form a clear solution, and the solution was placed at 5° C. and kept stirring, white turbidity appeared; the solid was filtrated and vacuum dried at 60° C. for 16 hours, and abemaciclib mesylate Form 6 was obtained.

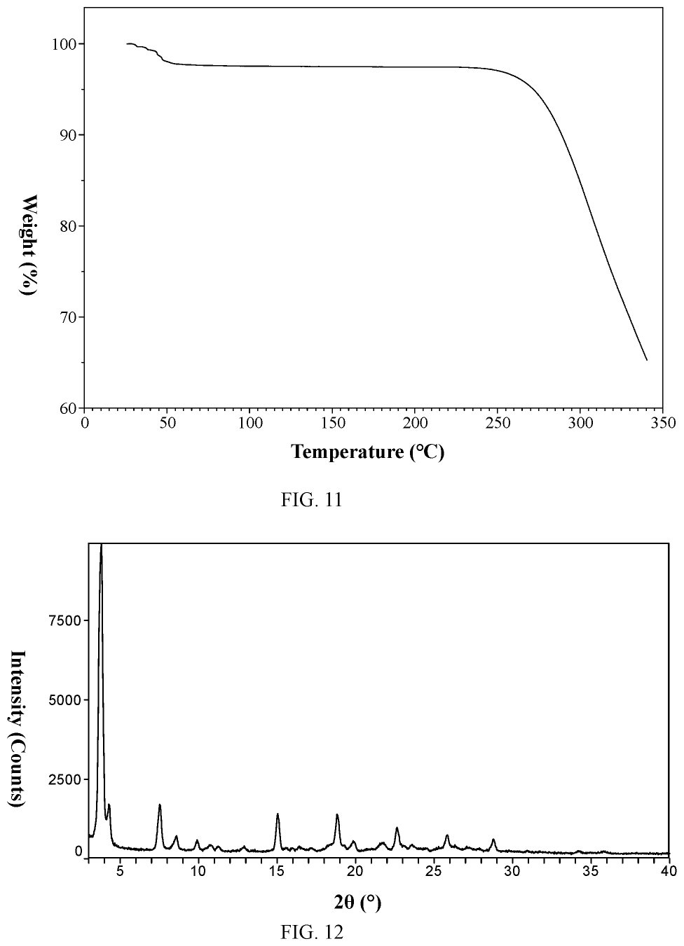

Its XRPD pattern is shown in FIG. 12.

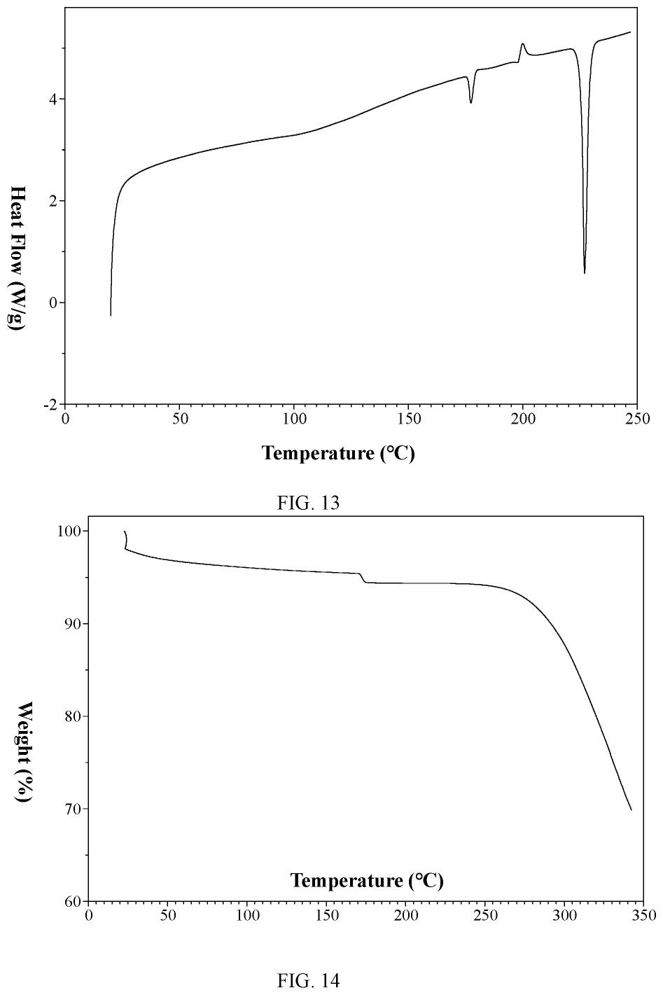

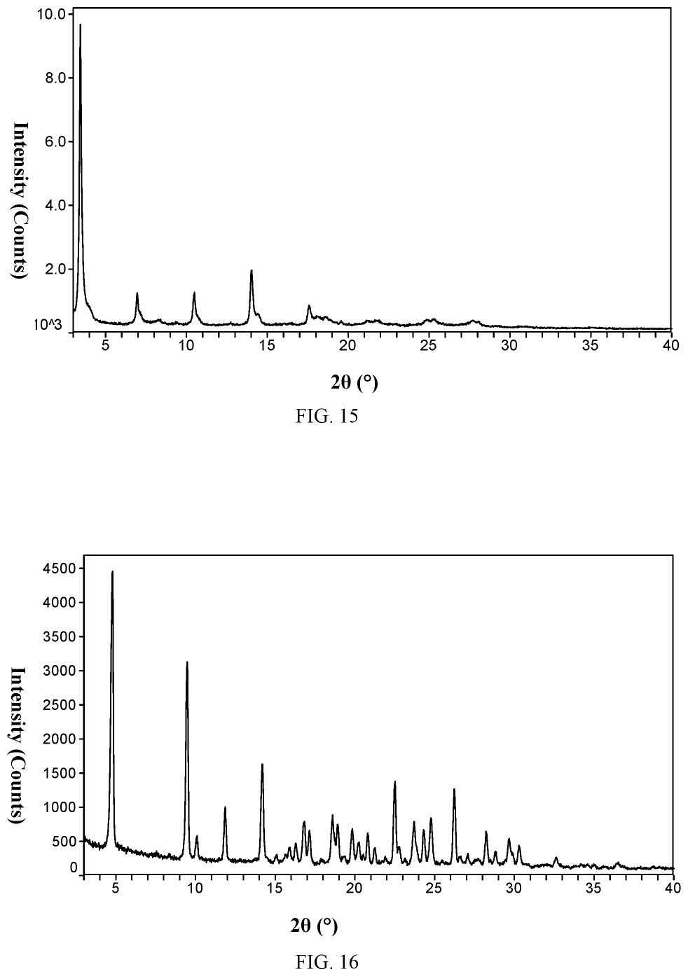

Its DSC thermogram is shown in FIG. 13.

Its TGA thermogram is shown in FIG. 14.

### Example 25

20 mg abemaciclib mesylate in Preparation Example 2 was dissolved in 10 mL ethanol, the solution was heated at 60° C. to form a clear solution, and the solution was placed at −10° C. and kept stirred, white turbidity appeared; the solid was filtrated and vacuum dried at 25° C. for 48 hours, and abemaciclib mesylate Form 6 was obtained.

### Example 26

20 mg abemaciclib mesylate in Preparation Example 2 was placed in an environment at 20° C. and 58% RH for 7 days, and abemaciclib mesylate Form 6 was obtained.

### Example 27

20 mg abemaciclib mesylate in Preparation Example 2 was placed in an environment at 20° C. and 58% RH for 1 day, and abemaciclib mesylate Form 6 was obtained.

### Example 28

20 mg abemaciclib mesylate in Preparation Example 2 was placed in an environment at 40° C. and 75% RH for 1 day, and abemaciclib mesylate Form 6 was obtained.

The samples prepared in Examples 25 to 28 have essentially the same or similar XRPD patterns (not shown), TGA thermograms (not shown) as those of the sample of Example 24, indicating that the samples of Examples 25 to 28 and Example 24 have the same crystalline form.

### Example 29

50 mg abemaciclib mesylate Form 1 in Example 1 was placed at 210° C. for 5 minutes, and abemaciclib mesylate Form 8 was obtained.

Its XRPD pattern is shown in FIG. 16.

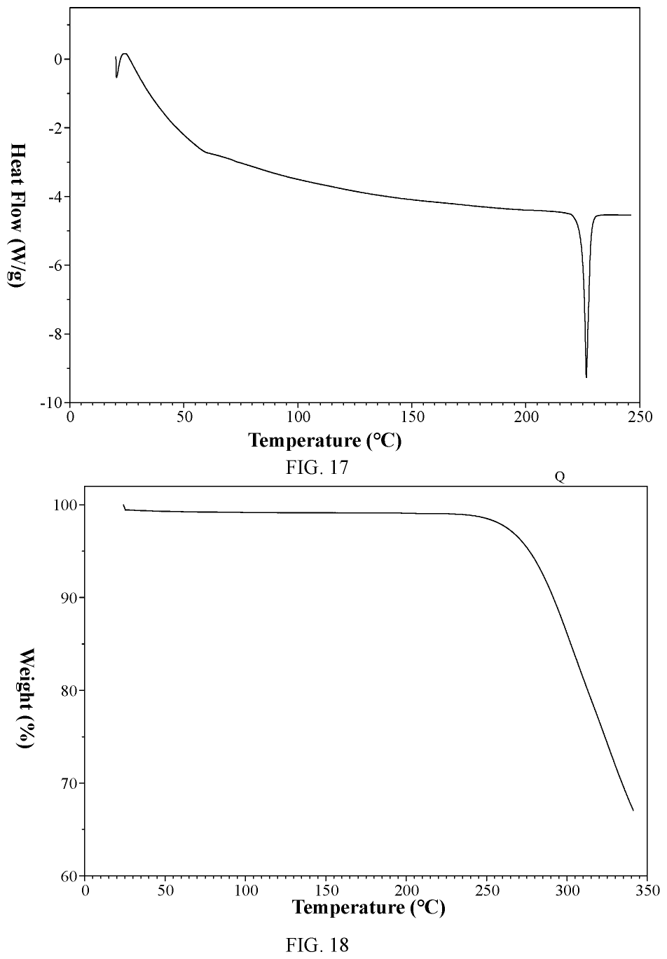

Its DSC thermogram is shown in FIG. 17.

Its TGA thermogram is shown in FIG. 18.

### Example 30

50 mg abemaciclib mesylate Form 2 in Example 7 was placed at 200° C. for 8 minutes, and abemaciclib mesylate Form 8 was obtained.

### Example 31

50 mg abemaciclib mesylate Form 5 in Example 10 was at 210° C. for 5 minutes, and abemaciclib mesylate Form 8 was obtained.

### Example 32

50 mg abemaciclib mesylate Form 6 in Example 24 was placed at 200° C. for 10 minutes, and abemaciclib mesylate Form 8 was obtained.

The samples prepared in Examples 30 to 32 have essentially the same or similar XRPD patterns (not shown), TGA thermograms (not shown) as those of the sample of Example 29, indicating that the samples of Examples 30 to 33 and Example 29 have the same crystalline form.

### Example 33

| Components | Dosage (mg) |
|---|---|
| Abemaciclib mesylate Form 1 | 119 |
| Starch | 158 |
| Hydroxypropyl cellulose | 30 |
| Magnesium stearate | 6 |
| Talcum powder | 6 |
| Total | 319 |

Abemaciclib mesylate Form 1, starch, hydroxypropyl cellulose, magnesium stearate and talcum powder were mixed and filled into the capsule.

### Example 34

| Components | Dosage (mg) |
|---|---|
| Abemaciclib mesylate Form 1 | 119 |
| Hydroxypropyl cellulose | 30 |
| Starch | 158 |
| Magnesium stearate | 6 |
| Talcum powder | 6 |
| Total | 319 |

Hydroxypropyl cellulose aqueous solution, abemaciclib mesylate Form 1 and starch were wet granulated, dried and mixed with magnesium stearate and talcum powder and then compressed into tablets.

### Examples 35 to 41

The abemaciclib mesylate Form 1 in Example 33 and Example 34 was replaced with abemaciclib mesylate Form 2, Form 3, Form 4, Form 5, Form 6, Form 7 and Form 8 in present invention, respectively, the free base in these formulations has the same molar amount as that of the free base in the abemaciclib mesylate Form 1 of the present invention, the other components in each formulation were the same as those in Examples 33 and 34, and the preparation steps of each preparation were the same as those in Examples 33 and 34.

### Comparative Example 1

Form stability experiment of crystalline forms: Placed 20 mg abemaciclib mesylate in Preparation Example 1, abemaciclib mesylate Form 1, Form 2, Form 5, Form 6, and Form 8 of the present invention at 60° C. or at room temperature under dry conditions for 30 days, then carried out XRD crystalline form test.

| Crystalline form | Test result after 30 day |
|---|---|
| Abemaciclib mesylate | Amorphous |
| Abemaciclib mesylate Form 1 | Form 1 |
| Abemaciclib mesylate Form 2 | Form 2 |
| Abemaciclib mesylate Form 5 | Form 5 |
| Abemaciclib mesylate Form 6 | Form 6 |
| Abemaciclib mesylate Form 8 | Form 8 |

The results showed that, after having been placed at 60° C. or room temperature under dry conditions for 30 days, abemaciclib mesylate Form 1, Form 2, Form 5, Form 6, and Form 8 of the present invention still kept their original crystalline forms. It indicated that they have better form stability than the prior art.

All the patents, patent application disclosures, patent applications and non-patented publications cited in this document are incorporated into this document by citation in full.

The above mentioned are only the embodiments of the present invention, which do not cover the entire protection scope of the present invention. Within the technical scope revealed in the present invention, modifications or replacements made by those skilled in the art without creative labor should all be within the protection scope of the present invention. Therefore, these shall be made within the scope of the present invention defined by the claims.

What is claimed is:

1. A crystalline abemaciclib mesylate having the structure shown below: 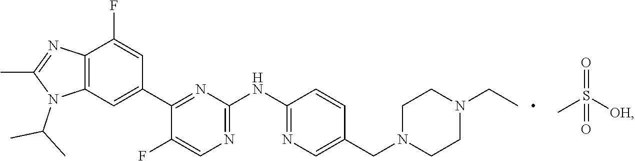
wherein the crystalline abemaciclib mesylate is anhydrous, a hydrate, or a non-solvate.

2. The crystalline abemaciclib mesylate according to claim 1, wherein the crystalline abemaciclib mesylate is crystalline abemaciclib mesylate Form 1, and wherein, measured using Cu-Kα radiation, the X-ray powder diffraction pattern of the crystalline abemaciclib mesylate Form 1, expressed as 2θ angles, has the following characteristic peaks: 4.0°±0.2°, 8.2°±0.2°, 23.6°±0.2° and 26.9°±0.2°.

3. The crystalline abemaciclib mesylate according to claim 2, wherein, measured using Cu-Kα radiation, the X-ray powder diffraction pattern of the crystalline abemaciclib mesylate Form 1, expressed as 2θ angles, has one or more of the following characteristic peaks: 15.9°±0.2°, 16.6°±0.2°, 17.0°±0.2°, 18.5°±0.2° and 20.7°±0.2°.

4. The crystalline abemaciclib mesylate according to claim 2 or 3, wherein the X-ray powder diffraction pattern of the crystalline abemaciclib mesylate Form 1, expressed as 2θ angles, also has one or more of the following characteristic peaks: 4.7°±0.2°, 9.5°±0.2°, 11.4°±0.2°, 12.7°±0.2°, 19.3°±0.2°, 22.5°±0.2° and 26.3°±0.2°.

5. The crystalline abemaciclib mesylate according to any one of claims 2 to 4, wherein the X-ray powder diffraction pattern of the crystalline abemaciclib mesylate Form 1, expressed as 2θ angles, further has one or more of the following characteristic peaks: 10.5°±0.2°, 14.3°±0.2°, 15.3°±0.2°, 17.9°±0.2°, 18.3°±0.2°, 19.9°±0.2°, 20.2°±0.2°, 29.7°±0.2° and 30.5°±0.2°.

6. A method of preparing the crystalline abemaciclib mesylate Form 1 according to any one of claims 2 to 5, comprising any one of the following methods:
1) dissolving abemaciclib in a haloalkane solvent to form a clear solution, stirring and dripping in methanesulfonic acid, adding an anti-solvent while stirring, filtrating and drying the solid to obtain the crystalline abemaciclib mesylate Form 1:
preferably, the haloalkane solvent is dichloromethane;
preferably, the molar ratio of abemaciclib and methanesulfonic acid is 1:1 to 1:1.5;
preferably, the anti-solvent is isopropyl ether in an amount is 2 to 4 times that of dichloromethane;
preferably, the mass-to-volume ratio of abemaciclib to the solvent is 20 to 170 mg:1 mL, more preferably, 40 to 170 mg:1 mL;
preferably, the reaction is carried out at room temperature;
preferably, the stirring crystallization time is 3 to 5 days;
preferably, the drying temperature is 25 to 60° C.;
preferably, the drying time is 16 to 48 hours; or
2) dissolving abemaciclib in a haloalkane solvent to form a clear solution, stirring and then dripping in methanesulfonic acid, stirring, filtrating and drying the solid to obtain the crystalline abemaciclib mesylate Form 1:
preferably, the haloalkane solvent is dichloromethane;
preferably, the mole ratio of abemaciclib and methanesulfonic acid is 1:1 to 1:1.5;
preferably, the mass-to-volume ratio of abemaciclib to the solvent is 50 to 250 mg:1 mL, more preferably, 60 to 250 mg:1 mL;
preferably, the reaction is carried out at room temperature;
preferably, the stirring crystallization time is 3 to 7 days;
preferably, the drying temperature is 25 to 60° C.;
preferably, the drying time is 16 to 48 hours.

7. The crystalline abemaciclib mesylate according to claim 1, wherein the crystalline abemaciclib mesylate is crystalline abemaciclib mesylate Form 2, and wherein, measured using Cu-Kα radiation, the X-ray powder diffraction pattern of the crystalline abemaciclib mesylate Form 2, expressed as 2θ angles, has the following characteristic peaks: 6.1°±0.2°, 12.0°±0.2°, 14.3°±0.2° and 21.6°±0.2°.

8. The crystalline abemaciclib mesylate according to claim 7, wherein the X-ray powder diffraction pattern of the crystalline abemaciclib mesylate Form 2, expressed as 2θ angles, further has one or more of the following characteristic peaks: 11.4°±0.2°, 12.7±0.2°, 13.0±0.2°, 14.7±0.2°, 15.6±0.2°, 17.5°±0.2°, 22.8°±0.2°, 23.4°±0.2°, 24.6°±0.2° and 26.9°±0.2°.

9. The crystalline abemaciclib mesylate according to claim 7 or 8, wherein the X-ray powder diffraction pattern of the crystalline abemaciclib mesylate Form 2, expressed as 2θ angles, further has one or more of the following characteristic peaks: 11.2±0.2°, 15.1±0.2°, 18.0±0.2°, 20.0±0.2°, 23.1°±0.2°, 24.4°±0.2°, 25.7±0.2°, 27.5±0.2°, 28.6±0.2°, 29.6±0.2°, 30.2±0.2° and 32.6°±0.2°.

10. A method of preparing the crystalline abemaciclib mesylate Form 2 according to any one of claims 7 to 9, the method comprising:
dissolving abemaciclib in a ketone solvent to form a suspension, stirring and then dripping in methanesulfonic acid, stirring, filtrating and drying the solid to obtain the crystalline abemaciclib mesylate Form 2:
preferably, the ketone solvent is acetone;
preferably, the molar ratio of abemaciclib and methanesulfonic acid is 1:1 to 1:1.5;
preferably, the mass-to-volume ratio of abemaciclib to the solvent is 5 to 25 mg:1 mL, more preferably, 10 to 25 mg:1 mL;
preferably, the reaction is carried out at room temperature;
preferably, the stirring crystallization time is 3 to 7 days;
preferably, the drying temperature is 25 to 60° C.;
preferably, the drying time is 16 to 48 hours.

11. The crystalline abemaciclib mesylate according to claim 1, wherein the crystalline abemaciclib mesylate is crystalline abemaciclib mesylate Form 5, and wherein, measured using Cu-Kα radiation, the X-ray powder diffraction pattern of the crystalline abemaciclib mesylate Form 5, expressed as 2θ angles, has the following characteristic peaks: 4.4°±0.2°, 9.0°±0.2°, 18.1±0.2° and 23.0°±0.2°.

12. The crystalline abemaciclib mesylate according to claim 11, wherein the X-ray powder diffraction pattern of the crystalline abemaciclib mesylate Form 5, expressed as 2θ angles, further has one or more of the following characteristic peaks: 11.8°±0.2°, 13.2±0.2°, 13.5°±0.2°, 16.3±0.2°, 20.6±0.2°, 21.7°±0.2°, 26.7°±0.2° and 30.7°±0.2°.

13. The crystalline abemaciclib mesylate according to claim 11 or 12, wherein, the X-ray powder diffraction pattern of the crystalline abemaciclib mesylate Form 5, expressed as 2θ angles, further has one or more of the following characteristic peaks: 14.2±0.2°, 14.5±0.2°, 15.4±0.2°, 17.1±0.2°, 18.2°±0.2°, 19.2°±0.2° and 22.1°±0.2°.

14. A method of preparing the crystalline abemaciclib mesylate Form 5 according to any one of claims 11 to 13, comprising any one of the following methods:
1) dissolving abemaciclib mesylate in a C₁ to C₄ alcohol, a C₃ to C₆ ether, a C₄ to C₅ ester, a C₃ to C₄ ketone, a cycloether, nitrile, water, an alkane, nitromethane, or any of mixtures thereof to form a suspension, stirring, separating and drying the solid to obtain the crystalline abemaciclib mesylate Form 5;
preferably, the mass-to-volume ratio of abemaciclib mesylate to the solvent is 15 to 100 mg:1 mL, more preferably, 20 to 50 mg:1 mL;
preferably, the reaction is carried out at 4 to 40° C.;
preferably, the stirring crystallization time is 3 to 7 days;
preferably, the drying temperature is 25 to 60° C.;
preferably, the drying time is 16 to 48 hours; or
2) dissolving abemaciclib mesylate in a C₁ to C₄ alcohol to form a clear solution, adding an anti-solvent while stirring, filtrating and drying the solid to obtain the crystalline abemaciclib mesylate Form 5;
preferably, the mass-to-volume ratio of abemaciclib mesylate to the C₁ to C₄ alcohol is 10 to 35 mg:1 mL, more preferably, 10 to 20 mg:1 mL;
preferably, the anti-solvent is selected from a C₃ to C₆ ether or a C₆ to C₇ alkane, in an amount 2 to 4 times that of the C₁ to C₄ alcohol;
preferably, the reaction is carried out at room temperature;
preferably, the drying temperature is 25 to 60° C.;
preferably, the drying time is 16 to 48 hours.

15. The crystalline abemaciclib mesylate according to claim 1, wherein the crystalline abemaciclib mesylate is crystalline abemaciclib mesylate Form 6, and wherein, measured using Cu-Kα radiation, the X-ray powder diffraction pattern of the crystalline abemaciclib mesylate Form 6, expressed as 2θ angles, has the following characteristic peaks: 3.8°±0.2°, 7.5°±0.2°, 15.0°±0.2° and 18.8°±0.2°.

16. The crystalline abemaciclib mesylate according to claim 15, wherein the X-ray powder diffraction pattern of the crystalline abemaciclib mesylate Form 6, expressed as 2θ angles, further has one or more of the following characteristic peaks: 4.3°±0.2°, 8.6°±0.2°, 9.9±0.2°, 10.8±0.2°, 11.2±0.2°, 19.9±0.2°, 21.8°±0.2°, 22.6°±0.2°, 25.8°±0.2° and 28.8°±0.2°.

17. The crystalline abemaciclib mesylate according to claim 15 or 16, wherein the X-ray powder diffraction pattern of the crystalline abemaciclib mesylate Form 6, expressed as 2θ angles, further has one or more of the following characteristic peaks: 12.9±0.2°, 16.4±0.2°, 17.2±0.2°, 18.3±0.2°, 19.2°±0.2°, 23.6°±0.2°, 24.5°±0.2°, 26.3°±0.2°, 27.2°±0.2°, 27.9°±0.2°, 34.2°±0.2° and 35.8°±0.2°.

18. A method of preparing the crystalline abemaciclib mesylate Form 6 according to any one of claims 15 to 17, comprising any one of the following methods:
1) placing solid abemaciclib mesylate at 58% to 85% relative humidity to obtain the crystalline abemaciclib mesylate Form 6;
preferably, the placing environment is at 20 to 40° C.; or
2) dissolving solid abemaciclib mesylate in ethanol, heating the solution to form a clear solution, stirring at low temperature, filtrating and drying the solid to obtain the crystalline abemaciclib mesylate Form 6;
preferably, the mass-to-volume ratio of solid abemaciclib mesylate to ethanol is 20 to 25 mg:1 mL;
preferably, the low temperature is −10 to 5° C.;
preferably, the drying temperature is 25 to 60° C.;
preferably, the drying time is 16 to 48 hours.

19. The crystalline abemaciclib mesylate according to claim 1, wherein the crystalline abemaciclib mesylate is crystalline abemaciclib mesylate Form 8, and wherein, measured using Cu-Kα radiation, the X-ray powder diffraction pattern of the crystalline abemaciclib mesylate Form 8, expressed as 2θ angles, has the following characteristic peaks: 4.8°±0.2°, 9.5°±0.2°, 14.2°±0.2° and 22.5±0.2°.

20. The crystalline abemaciclib mesylate according to claim 19, wherein the X-ray powder diffraction pattern of the crystalline abemaciclib mesylate Form 8, expressed as 2θ angles, further has one or more of the following characteristic peaks: 11.9°±0.2°, 16.8±0.2°, 18.6±0.2°, 23.7±0.2°, 24.8°±0.2° and 26.2°±0.2°.

21. The crystalline abemaciclib mesylate according to claim 19 or 20, wherein the X-ray powder diffraction pattern of the crystalline abemaciclib mesylate Form 8, expressed as 2θ angles, further has one or more of the following characteristic peaks: 10.1±0.2°, 17.1±0.2°, 18.9±0.2°, 19.8±0.2°, 20.3°±0.2°, 20.8°±0.2°, 24.3°±0.2°, 28.2°±0.2°, 29.7°±0.2° and 30.3°±0.2°.

22. A method of preparing the crystalline abemaciclib mesylate Form 8 according to any one of claims 19 to 21, the method comprising:
keeping the crystalline abemaciclib mesylate Form 1, Form 2, Form 5, or Form 6 at 200 to 210° C. for 5 to 15 minutes to obtain the crystalline abemaciclib mesylate Form 8.

23. A pharmaceutical composition comprising a therapeutically effective amount of the crystalline abemaciclib mesylate Form 1 according to any one of claims 2 to 6, the crystalline abemaciclib mesylate Form 2 according to any one of claims 7 to 10, the crystalline abemaciclib mesylate Form 5 according to any one of claims 11 to 14, the crystalline abemaciclib mesylate Form 6 according to any one of claims 15 to 18, or the crystalline abemaciclib mesylate Form 8 according to any one of claims 19 to 22, and at least one pharmaceutically acceptable carrier or excipient.

24. The pharmaceutical composition according to claim 23, wherein the pharmaceutical composition is in an orally administered form, specifically, tablet, capsule, granule, suppository, emulsion, suspension or solution.

25. The uses of the crystalline abemaciclib mesylate Form 1 according to any one of claims 2 to 6, the crystalline abemaciclib mesylate Form 2 according to any one of claims 7 to 10, the crystalline abemaciclib mesylate Form 5 according to any one of claims 11 to 14, the crystalline abemaciclib mesylate Form 6 according to any one of claims 15 to 18, or the crystalline abemaciclib mesylate Form 8 according to any one of claims 19 to 22, in the preparation of the drugs treating cancer diseases, such as colorectal cancer, breast cancer, lung cancer, prostate cancer, glioblastoma, mantle cell lymphoma, chronic myelocytic leukemia, and acute myelocytic leukemia, etc.

26. A method of treating colorectal cancer, breast cancer, lung cancer, prostate cancer, glioblastoma, mantle cell lymphoma, chronic myelocytic leukemia, or acute myelocytic leukemia, the method comprising providing the patients in need a therapeutically effective amount of the crystalline abemaciclib mesylate Form 1 according to any one of claims 2 to 6, the crystalline abemaciclib mesylate Form 2 according to any one of claims 7 to 10, the crystalline abemaciclib mesylate Form 5 according to any one of claims 11 to 14, the crystalline abemaciclib mesylate Form 6 according to any one of claims 15 to 18, or the crystalline abemaciclib mesylate Form 8 according to any one of claims 19 to 22, or a pharmaceutical composition according to any one of claims 23 to 25, wherein the cancer diseases comprise colorectal cancer, breast cancer, lung cancer, prostate cancer, glioblastoma, mantle cell lymphoma, chronic myelocytic leukemia, and acute myelocytic leukemia.
